SEQUENCE LISTING (6 sequences)

SEQ ID NO: 1 - protein, 452 aa, Homo sapiens
MEVTTRLTWNDENHLRKLLGNVSLSLLYKSSVHGGSIEDMVERCSRQGCTITMAYIDYNMIVAFMLGNYINLHESSTEPNDSLWFSLQKKNDTTEIETLLLNTAPKIIDEQLVCRLSKTDIFIICRDNKIYLDKMITRNLKLRFYGHRQYLECEVFRVEGIKDNLDDIKRIIKAREHRNRLLADIRDYRPYADLVSEIRILLVGPVGSGKSSFFNSVKSIFHGHVTGQAVVGSDITSITERYRIYSVKDGKNGKSLPFMLCDTMGLDGAEGAGLCMDDIPHILKGCMPDRYQFNSRKPITPEHSTFITSPSLKDRIHCVAYVLDINSIDNLYSKMLAKVKQVHKEVLNCGIAYVALLTKVDDCSEVLQDNFLNMSRSMTSQSRVMNVHKMLGIPISNILMVGNYASDLELDPMKDILILSALRQMLRAADDFLEDLPLEETGAIERALQPCI

SEQ ID NO: 2 - protein, 851 aa, Homo sapiens
MAQWEMLQNLDSPFQDQLHQLYSHSLLPVDIRQYLAVWIEDQNWQEAALGSDDSKATMLFFHFLDQLNYECGRCSQDPESLLLQHNLRKFCRDIQPFSQDPTQLAEMIFNLLLEEKRILIQAQRAQLEQGEPVLETPVESQQHEIESRILDLRAMMEKLVKSISQLKDQQDVFCFRYKIQAKGKTPSLDPHQTKEQKILQETLNELDKRRKEVLDASKALLGRLTTLIELLLPKLEEWKAQQQKACIRAPIDHGLEQLETWFTAGAKLLFHLRQLLKELKGLSCLVSYQDDPLTKGVDLRNAQVTELLQRLLHRAFVVETQPCMPQTPHRPLILKTGSKFTVRTRLLVRLQEGNESLTVEVSIDRNPPQLQGFRKFNILTSNQKTLTPEKGQSQGLIWDFGYLTLVEQRSGGSGKGSNKGPLGVTEELHIISFTVKYTYQGLKQELKTDTLPVVIISNMNQLSIAWASVLWFNLLSPNLQNQQFFSNPPKAPWSLLGPALSWQFSSYVGRGLNSDQLSMLRNKLFGQNCRTEDPLLSWADFTKRESPPGKLPFWTWLDKILELVHDHLKDLWNDGRIMGFVSRSQERRLLKKTMSGTFLLRFSESSEGGITCSWVEHQDDDKVLIYSVQPYTKEVLQSLPLTEIIRHYQLLTEENIPENPLRFLYPRIPRDEAFGCYYQEKVNLQERRKYLKHRLIVVSNRQVDELQQPLELKPEPELESLELELGLVPEPELSLDLEPLLKAGLDLGPELESVLESTLEPVIEPTLCMVSQTVPEPDQGPVSQPVPEPDLPCDLRHLNTEPMEIFRNCVKIEEIMPNGDPLLAGQNTVDEVYVSRPSHFYTDGPLMPSDF

SEQ ID NO: 3 - protein, 393 aa, Homo sapiens
MASGRARCTRKLRNWVVEQVESGQFPGVCWDDTAKTMFRIPWKHAGKQDFREDQDAAFFKAWAIFKGKYKEGDTGGPAVWKTRLRCALNKSSEFKEVPERGRMDVAEPYKVYQLLPPGIVSGQPGTQKVPSKRQHSSVSSERKEEEDAMQNCTLSPSVLQDSLNNEEEGASGGAVHSDIGSSSSSSSPEPQEVTDTTEAPFQGDQRSLEFLLPPEPDYSLLLTFIYNGRVVGEAQVQSLDCRLVAEPSGSESSMEQVLFPKPGPLEPTQRLLSQLERGILVASNPRGLFVQRLCPIPISWNAPQAPPGPGPHLLPSNECVELFRTAYFCRDLVRYFQGLGPPPKFQVTLNFWEESHGSSHTPQNLITVKMEQAFARYLLEQTPEQQAAILSLV

SEQ ID NO: 4 - protein, 232 aa, Homo sapiens
MATAESRALQFAEGAAFPAYRAPHAGGALLPPPSPAAALLPAPPAGPGPATFAGFLGRDPGPAPPPPASLGSPAPPKGAAAPSASQRRKRTSFSAEQLQLLELVFRRTRYPDIHLRERLAALTLLPESRIQVWFQNRRAKSRRQSGKSFQPLARPEIILNHCAPGTETKCLKPQLPLEVDVNCLPEPNGVGGGISDSSSQGQNFETCSPLSEDIGSKLDSWEEHIFSAFGNF

SEQ ID NO: 5 - protein, 1584 aa, Homo sapiens
MSKQVSLPEMIKDWTKEHVKKWVNEDLKINEQYGQILLSEEVTGLVLQELTEKDLVEMGLPWGPALLIKRSYNKLNSKSPESDNHDPGQLDNSKPSKTEHQKNPKHTKKEEENSMSSNIDYDPREIRDIKQEESILMKENVLDEVANAKHKKKGKLKPEQLTCMPYPFDQFHDSHRYIEHYTLQPETGALNLIDPIHEFKALTNTETATEVDIKMKFSNEVFRFASACMNSRTNGTIHFGVKDKPHGEIVGVKITSKAAFIDHFNVMIKKYFEESEINEAKKCIREPRFVEVLLQNNTPSDRFVIEVDTIPKHSICNDKYFYIQMQICKDKIWKQNQNLSLFVREGASSRDILANSKQRDVDFKAFLQNLKSLVASRKEAEEEYGMKAMKKESEGLKLVKLLIGNRDSLDNSYYDWYILVTNKCHPNQIKHLDFLKEIKWFAVLEFDPESMINGVVKAYKESRVANLHFPNQYEDKTTNMWEKISTLNLYQQPSWIFCNGRSDLKSETYKPLEPHLWQRERASEVRKLILFLTDENIMTRGKFLVVFLLLSSVESPGDPLIETFWAFYQALKGMENMLCISVNSHIYQRWKDLLQTRMKMEDELTNHSISTLNIELVNSTILKLKSVTRSSRRFLPARGSSSVILEKKKEDVLTALEILCENECTETDIEKDKSKFLEFKKSKEEHFYRGGKVSWWNFYFSSENYSSDFVKRDSYEKLKDLIHCWAESPKPIFAKIINLYHHPGCGGTTLAMHVLWDLKKNFRCAVLKNKTTDFAEIAEQVINLVTYRAKSHQDYIPVLLLVDDFEEQENVYFLQNAIHSVLAEKDLRYEKTLVIILNCMRSRNPDESAKLADSIALNYQLSSKEQRAFGAKLKEIEKQHKNCENFYSFMIMKSNFDETYIENVVRNILKGQDVDSKEAQLISFLALLSSYVTDSTISVSQCEIFLGIIYTSTPWEPESLEDKMGTYSTLLIKTEVAEYGRYTGVRIIHPLIALYCLKELERSYHLDKCQIALNILEENLFYDSGIGRDKFQHDVQTLLLTRQRKVYGDETDTLFSPLMEALQNKDIEKVLSAGSRRFPQNAFICQALARHFYIKEKDFNTALDWARQAKMKAPKNSYISDTLGQVYKSEIKWWLDGNKNCRSITVNDLTHLLEAAEKASRAFKESQRQTDSKNYETENWSPQKSQRRYDMYNTACFLGEIEVGLYTIQILQLTPFFHKENELSKKHMVQFLSGKWTIPPDPRNECYLALSKFTSHLKNLQSDLKRCFDFFIDYMVLLKMRYTQKEIAEIMLSKKVSRCFRKYTELFCHLDPCLLQSKESQLLQEENCRKKLEALRADRFAGLLEYLNPNYKDATTMESIVNEYAFLLQQNSKKPMTNEKQNSILANIILSCLKPNSKLIQPLTTLKKQLREVLQFVGLSHQYPGPYFLACLLFWPENQELDQDSKLIEKYVSSLNRSFRGQYKRMCRSKQASTLFYLGKRKGLNSIVHKAKIEQYFDKAQNTNSLWHSGDVWKKNEVKDLLRRLTGQAEGKLISVEYGTEEKIKIPVISVYSGPLRSGRNIERVSFYLGFSIEGPLAYDIEVI

SEQ ID NO: 6 - protein, 345 aa, Homo sapiens
MQRLVAWDPACLPLPPPPPAFKSMEVANFYYEADCLAAAYGGKAAPAAPPAARPGPRPPAGELGSIGDHERAIDFSPYLEPLGAPQAPAPATATDTFEAAPPAPAPAPASSGQHHDFLSDLFSDDYGGKNCKKPAEYGYVSLGRLGAAKGALHPGCFAPLHPPPPPPPPPAELKAEPGFEPADCKRKEEAGAPGGGAGMAAGFPYALRAYLGYQAVPSGSSGSLSTSSSSSPPGTPSPADAKAPPTACYAGAAPAPSQVKSKAKKTVDKHSDEYKIRRERNNIAVRKSRDKAKMRNLETQHKVLELTAENERLQKKVEQLSRELSTLRNLFKQLPEPLLASSGHC

(19) United States
(12) Patent Application Publication
(10) Pub. No.: US 2025/0283174 A1
(43) Pub. Date: Sep. 11, 2025

(54) METHODS FOR ASSESSING THE EXHAUSTION OF HEMATOPOIETIC STEM CELLS INDUCED BY CHRONIC INFLAMMATION

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); FONDATION IMAGINE, Paris (FR); UNIVERSITÉ PARIS CITÉ, Paris (FR)

(72) Inventors: Emmanuelle SIX, Paris (FR); Adeline DENIS, Paris (FR); Marina CAVAZZANA, Paris (FR); Agathe GUILLOUX, Paris (FR); Steicy SOBRINO, Paris (FR); Antonio RAUSELL DE FRIAS, Paris (FR); Akira CORTAL, Paris (FR); Loredana MARTIGNETTI, Paris cedex 05 (FR)

(21) Appl. No.: 18/862,787

(22) PCT Filed: May 15, 2023
(86) PCT No.: PCT/EP2023/062884

§ 371 Date: Nov. 4, 2024

(30) Foreign Application Priority Data
May 16, 2022 (EP) 22305722.5

(51) Int. Cl. C12Q 1/6883

(52) U.S. Cl. CPC C12Q 1/6883; C12Q 2600/106; C12Q 2600/158

(57) ABSTRACT

Engraftment of hematopoietic stems cells (HSCs) is a potentially life-saving treatment therapy for various conditions including genetic blood cell diseases. In particular, the use of allogeneic HSCs to treat genetic blood cell diseases has become a clinical standard: HSCs can be modified ex vivo and transferred back to the recipient to produce functional, terminally-differentiated cells. There is a medical need for methods for assessing the exhaustion of HSCs for optimizing their therapeutic use, in particular in the context of gene therapy. The inventors performed a clinical trial of lentivirus-based gene therapy for the treatment of X-linked chronic granulomatous disease. Two patients showed stable engraftment and clinical benefits, whereas the other two progressively lost gene-corrected cells. Single-cell transcriptomic analysis revealed a significantly lower frequency of the most immature hematopoietic stem cell (HSC) in CGD patients, more pronounced in patients with defective engraftment. The two patients with defective engraftment presented a profound change in HSC status, a high interferon score, and elevated myeloid progenitor counts. The inventors used elastic-net logistic regression to identify a set of interferon genes and transcription factors that predicted the failure of HSC engraftment. They identified a set of 51 interferon genes and transcription factors that were upregulated specifically in P2 and P5 (including IFI44L, STAT2, IRF9, MX1, SAMD9L, and CEBPB) and that appeared to be predictive of defective HSC engraftment. Accordingly, the identified biomarkers can be very suitable for assessing the exhaustion of hematopoietic stems cells.

### FIELD OF THE INVENTION

The present invention is in the field of medicine, in particular haematology.

### BACKGROUND OF THE INVENTION

Hematopoietic stems cells, or “HSCs” are defined by their ability to self-renew and differentiate to replenish all blood lineages throughout adult life. Under homeostasis, the majority of HSCs are quiescent, and few stem cells are cycling to sustain haematopoiesis. HSCs are thus the most important element for establishing the long-term engraftment of hematopoietic transplants in recipients. Engraftment of HSCs is indeed a potentially life-saving treatment therapy for haematological malignancies such as leukemia and other diseases of the blood and immune system which include, but are not limited to, cancers (e.g., leukemia, lymphoma), blood disorders (e.g., inherited anemia, inborn errors of metabolism, aplastic anemia, beta-thalassemia, Blackfan-Diamond syndrome, globoid cell leukodystrophy, sickle cell anemia, severe combined immunodeficiency, X-linked lymphoproliferative syndrome, Wiskott-Aldrich syndrome, Hunter's syndrome, Hurler's syndrome Lesch Nyhan syndrome, osteopetrosis), chemotherapy rescue of the immune system, and other diseases (e.g., autoimmune diseases, diabetes, rheumatoid arthritis, system lupus erythromatosis). In particular, the use of allogeneic HSCs to treat genetic blood cell diseases has become a clinical standard: HSCs can be modified ex vivo and transferred back to the recipient to produce functional, terminally-differentiated cells. Recent evidence suggests that stress conditions such as chronic inflammation accelerate functional exhaustion of HSCs including their ability to repopulate and produce mature cells and thus their ability to be engrafted. There is thus a medical need for methods for assessing the exhaustion of HSCs in the context of chronic inflammation for optimizing their therapeutic use, in particular in the context of gene therapy.

### SUMMARY OF THE INVENTION

The present invention is defined by the claims. In particular, the present invention relates to methods for assessing for assessing the exhaustion of HSCs.

### DETAILED DESCRIPTION OF THE INVENTION

### Main Definitions

As used herein, the term “hematopoietic stem cell” or “HSC” refers to blood cells that have the capacity to self-renew and to differentiate into progenitors of blood cells. These progenitor cells are immature blood cells that cannot self-renew and must differentiate into mature blood cells. Hematopoietic stem and progenitor cells (HSPC) encompassed HSC and the downstream progenitors, they display a number of phenotypes, such as Lin-CD34+CD38−CD90+CD45RA− (HSC), Lin−CD34+CD38−CD90−CD45RA− (MPP), Lin−CD34+CD38+IL-3aloCD45RA−(CMP), and Lin−CD34+CD38+CD10+(BNKP) (Daley et al., Focus 18:62-67, 1996; Pimentel, E., Ed., Handbook of Growth Factors Vol. III: Hematopoietic Growth Factors and Cytokines, pp. 1-2, CRC Press, Boca Raton, Fla., 1994). Within the bone marrow microenvironment, the stem cells self-renew and maintain continuous production of hematopoietic stem cells that give rise to all mature blood cells throughout life. In some embodiments, the hematopoietic progenitor cells or hematopoietic stem cells are isolated from peripheral blood cells.

As used herein, the term “exhaustion” refers to the quantitative and qualitative decline in stem cell function. Stem cell function of hematopoietic stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal (which refers to the ability of hematopoietic stem cells to give rise to daughter cells that have equivalent potential as the mother cell, and further that this ability can repeatedly occur throughout the lifetime of an individual without exhaustion), and 3) the ability of hematopoietic stem cells or progeny thereof to be reintroduced into a transplant recipient whereupon they home to the hematopoietic stem cell niche and re-establish productive and sustained hematopoiesis. In particular, exhaustion results in a lower engraftment ability.

As used herein, the term “population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched.

As used herein, the term “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, the term “IFI44L” has its general meaning in the art and refers to the interferon-induced protein 44-like encoded by the IFI44L gene. An exemplary amino acid sequence for IFI44L is represented by SEQ ID NO:1.

>sp|Q53G44|IF44L_HUMAN Interferon-induced protein
44-like OS = Homo sapiens OX = 9606 GN = IFI44L
PE = 2 SV = 3
SEQ ID NO: 1
MEVTTRLTWNDENHLRKLLGNVSLSLLYKSSVHGGSIEDMVERCSRQGC
TITMAYIDYNMIVAFMLGNYINLHESSTEPNDSLWFSLQKKNDTTEIET
LLLNTAPKIIDEQLVCRLSKTDIFIICRDNKIYLDKMITRNLKLRFYGH
RQYLECEVFRVEGIKDNLDDIKRIIKAREHRNRLLADIRDYRPYADLVS
EIRILLVGPVGSGKSSFFNSVKSIFHGHVTGQAVVGSDITSITERYRIY
SVKDGKNGKSLPFMLCDTMGLDGAEGAGLCMDDIPHILKGCMPDRYQFN
SRKPITPEHSTFITSPSLKDRIHCVAYVLDINSIDNLYSKMLAKVKQVH
KEVLNCGIAYVALLTKVDDCSEVLQDNFLNMSRSMTSQSRVMNVHKMLG
IPISNILMVGNYASDLELDPMKDILILSALRQMLRAADDFLEDLPLEET
GAIERALQPCI

As used herein, the term “STAT2” has its general meaning in the art and refers to the signal transducer and activator of transcription 2 encode by the STAT2 gene. An exemplary amino acid sequence for STAT2 is represented by SEQ ID NO:2.

>sp|P52630|STAT2_HUMAN Signal transducer and
activator of transcription 2 OS = Homo sapiens
OX = 9606 GN = STAT2 PE = 1 SV = 1
SEQ ID NO: 2
MAQWEMLQNLDSPFQDQLHQLYSHSLLPVDIRQYLAVWIEDQNWQEAAL
GSDDSKATMLFFHFLDQLNYECGRCSQDPESLLLQHNLRKFCRDIQPFS
QDPTQLAEMIFNLLLEEKRILIQAQRAQLEQGEPVLETPVESQQHEIES
RILDLRAMMEKLVKSISQLKDQQDVFCFRYKIQAKGKTPSLDPHQTKEQ
KILQETLNELDKRRKEVLDASKALLGRLTTLIELLLPKLEEWKAQQQKA
CIRAPIDHGLEQLETWFTAGAKLLFHLRQLLKELKGLSCLVSYQDDPLT
KGVDLRNAQVTELLQRLLHRAFVVETQPCMPQTPHRPLILKTGSKFTVR
TRLLVRLQEGNESLTVEVSIDRNPPQLQGFRKFNILTSNQKTLTPEKGQ
SQGLIWDFGYLTLVEQRSGGSGKGSNKGPLGVTEELHIISFTVKYTYQG
LKQELKTDTLPVVIISNMNQLSIAWASVLWFNLLSPNLQNQQFFSNPPK
APWSLLGPALSWQFSSYVGRGLNSDQLSMLRNKLFGQNCRTEDPLLSWA
DFTKRESPPGKLPFWTWLDKILELVHDHLKDLWNDGRIMGFVSRSQERR
LLKKTMSGTFLLRFSESSEGGITCSWVEHQDDDKVLIYSVQPYTKEVLQ
SLPLTEIIRHYQLLTEENIPENPLRFLYPRIPRDEAFGCYYQEKVNLQE
RRKYLKHRLIVVSNRQVDELQQPLELKPEPELESLELELGLVPEPELSL
DLEPLLKAGLDLGPELESVLESTLEPVIEPTLCMVSQTVPEPDQGPVSQ
PVPEPDLPCDLRHLNTEPMEIFRNCVKIEEIMPNGDPLLAGQNTVDEVY
VSRPSHFYTDGPLMPSDF

As used herein, the term “IRF9” has its general meaning in the art and refers to the interferon regulatory factor 9 encoded by the IRF9 gene. An exemplary amino acid sequence for IRF9 is represented by SEQ ID NO:3.

>sp|Q00978|IRF9_HUMAN Interferon regulatory
factor 9 OS = Homo sapiens OX = 9606
GN = IRF9 PE = 1 SV = 1
SEQ ID NO: 3
MASGRARCTRKLRNWVVEQVESGQFPGVCWDDTAKTMFRIPWKHAGKQD
FREDQDAAFFKAWAIFKGKYKEGDTGGPAVWKTRLRCALNKSSEFKEVP
ERGRMDVAEPYKVYQLLPPGIVSGQPGTQKVPSKRQHSSVSSERKEEED
AMQNCTLSPSVLQDSINNEEEGASGGAVHSDIGSSSSSSSPEPQEVTDT
TEAPFQGDQRSLEFLLPPEPDYSLLLTFIYNGRVVGEAQVQSLDCRLVA
EPSGSESSMEQVLFPKPGPLEPTQRLLSQLERGILVASNPRGLFVQRLC
PIPISWNAPQAPPGPGPHLLPSNECVELFRTAYFCRDLVRYFQGLGPPP
KFQVTLNFWEESHGSSHTPQNLITVKMEQAFARYLLEQTPEQQAAILSL
V

As used herein, the term “MIX1” has its general meaning in the art and refers to the homeobox protein MIXL1 encoded by the MIX1 gene. An exemplary amino acid sequence for MIX1 is represented by SEQ ID NO:4.

>sp|Q9H2W2|MIXL1_HUMAN Homeobox protein MIXL1
OS = Homo sapiens OX = 9606 GN = MIXL1 PE = 1
SV = 1
SEQ ID NO: 4
MATAESRALQFAEGAAFPAYRAPHAGGALLPPPSPAAALLPAPPAGPGP
ATFAGELGRDPGPAPPPPASLGSPAPPKGAAAPSASQRRKRTSFSAEQL
QLLELVERRTRYPDIHLRERLAALTLLPESRIQVWFQNRRAKSRRQSGK
SFQPLARPEIILNHCAPGTETKCLKPQLPLEVDVNCLPEPNGVGGGISD
SSSQGQNFETCSPLSEDIGSKLDSWEEHIFSAFGNF

As used herein, the term ‘SAMD9L” has its general meaning in the art and refers to the sterile alpha motif domain-containing protein 9-like encoded by the SAMD9L gene. An exemplary amino acid sequence for SAMD9L is represented by SEQ ID NO:5.

>sp|Q8IVG5|SAM9L_HUMAN Sterile alpha motif
domain-containing protein 9-like OS = Homo
sapiens OX = 9606 GN = SAMD9L PE = 1 SV = 2
SEQ ID NO: 5
MSKQVSLPEMIKDWTKEHVKKWVNEDLKINEQYGQILLSEEVTGLVLQE
LTEKDLVEMGLPWGPALLIKRSYNKLNSKSPESDNHDPGQLDNSKPSKT
EHQKNPKHTKKEEENSMSSNIDYDPREIRDIKQEESILMKENVLDEVAN
AKHKKKGKLKPEQLTCMPYPFDQFHDSHRYIEHYTLQPETGALNLIDPI
HEFKALTNTETATEVDIKMKFSNEVFRFASACMNSRTNGTIHFGVKDKP
HGEIVGVKITSKAAFIDHFNVMIKKYFEESEINEAKKCIREPRFVEVLL
QNNTPSDRFVIEVDTIPKHSICNDKYFYIQMQICKDKIWKQNQNLSLFV
REGASSRDILANSKQRDVDFKAFLQNLKSLVASRKEAEEEYGMKAMKKE
SEGLKLVKLLIGNRDSLDNSYYDWYILVTNKCHPNQIKHLDFLKEIKWF
AVLEFDPESMINGVVKAYKESRVANLHFPNQYEDKTTNMWEKISTLNLY
QQPSWIFCNGRSDLKSETYKPLEPHLWQRERASEVRKLILFLTDENIMT
RGKFLVVFLLLSSVESPGDPLIETFWAFYQALKGMENMLCISVNSHIYQ
RWKDLLQTRMKMEDELTNHSISTLNIELVNSTILKLKSVTRSSRRFLPA
RGSSSVILEKKKEDVLTALEILCENECTETDIEKDKSKFLEFKKSKEEH
FYRGGKVSWWNFYFSSENYSSDFVKRDSYEKLKDLIHCWAESPKPIFAK
IINLYHHPGCGGTTLAMHVLWDLKKNFRCAVLKNKTTDFAEIAEQVINL
VTYRAKSHQDYIPVLLLVDDFEEQENVYFLQNAIHSVLAEKDLRYEKTL
VIILNCMRSRNPDESAKLADSIALNYQLSSKEQRAFGAKLKEIEKQHKN
CENFYSFMIMKSNFDETYIENVVRNILKGQDVDSKEAQLISFLALLSSY
VTDSTISVSQCEIFLGIIYTSTPWEPESLEDKMGTYSTLLIKTEVAEYG
RYTGVRIIHPLIALYCLKELERSYHLDKCQIALNILEENLFYDSGIGRD
KFQHDVQTLLLTRQRKVYGDETDTLFSPLMEALQNKDIEKVLSAGSRRF
PQNAFICQALARHFYIKEKDFNTALDWARQAKMKAPKNSYISDTLGQVY
KSEIKWWLDGNKNCRSITVNDLTHLLEAAEKASRAFKESQRQTDSKNYE
TENWSPQKSQRRYDMYNTACFLGEIEVGLYTIQILQLTPFFHKENELSK
KHMVQFLSGKWTIPPDPRNECYLALSKFTSHLKNLQSDLKRCFDFFIDY
MVLLKMRYTQKEIAEIMLSKKVSRCFRKYTELFCHLDPCLLQSKESQLL
QEENCRKKLEALRADRFAGLLEYLNPNYKDATTMESIVNEYAFLLQQNS
KKPMTNEKQNSILANIILSCLKPNSKLIQPLTTLKKQLREVLQFVGLSH
QYPGPYFLACLLFWPENQELDQDSKLIEKYVSSLNRSFRGQYKRMCRSK
QASTLFYLGKRKGLNSIVHKAKIEQYFDKAQNTNSLWHSGDVWKKNEVK
DLLRRLTGQAEGKLISVEYGTEEKIKIPVISVYSGPLRSGRNIERVSFY
LGFSIEGPLAYDIEVI

As used herein, the term “CEBPB” has its general meaning in the art and refers to the CCAAT/enhancer-binding protein beta encoded by the CEBPB gene. An exemplary amino acid sequence for CEBPB is represented by SEQ ID NO:6.

>sp|P17676|CEBPB_HUMAN CCAAT/enhancer-binding
protein beta OS = Homo sapiens OX = 9606
GN = CEBPB PE = 1 SV = 2
SEQ ID NO: 6
MQRLVAWDPACLPLPPPPPAFKSMEVANFYYEADCLAAAYGGKAAPAAP
PAARPGPRPPAGELGSIGDHERAIDFSPYLEPLGAPQAPAPATATDTFE
AAPPAPAPAPASSGQHHDFLSDLFSDDYGGKNCKKPAEYGYVSLGRLGA
AKGALHPGCFAPLHPPPPPPPPPAELKAEPGFEPADCKRKEEAGAPGGG
AGMAAGFPYALRAYLGYQAVPSGSSGSLSTSSSSSPPGTPSPADAKAPP
TACYAGAAPAPSQVKSKAKKTVDKHSDEYKIRRERNNIAVRKSRDKAKM
RNLETQHKVLELTAENERLQKKVEQLSRELSTLRNLFKQLPEPLLASSG
HC

As used herein, the term “predetermined reference value” refers to the expression level of one gene determined in a population of HSC obtained from the general population or from a selected population of subjects. Typically, the predetermined reference value is a threshold value or a cut-off value that can be determined experimentally, empirically, or theoretically. A threshold value can also be arbitrarily selected based upon the existing experimental and/or clinical conditions, as would be recognized by a person of ordinary skilled in the art. For example, retrospective measurement of expression levels in properly banked historical patient samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after quantifying the expression level in a group of reference, one can use algorithmic analysis for the statistic treatment of the determined levels in samples to be tested, and thus obtain a classification standard having significance for sample classification. The full name of ROC curve is Receiver Operator Characteristic Curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “therapeutically effective amount” is meant a sufficient amount of population of HSCs to treat the disease at a reasonable benefit/risk ratio applicable to any medical treatment. It will be understood that the total usage compositions of the present invention will be decided by the attending physician within the scope of sound medical judgment. The specific therapeutically effective dose level for any particular patient will depend upon a variety of factors including the age, body weight, general health, sex and diet of the patient, the time of administration, route of administration, the duration of the treatment, drugs used in combination or coincidental with the population of HSCs, and like factors well known in the medical arts. In some embodiments, the HSCs are formulated by first harvesting them from their culture medium, and then washing and concentrating the HSCs in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of HSCs in the composition is dependent on the relative representation of the HSCs with the desired specificity, on the age and weight of the recipient, and on the severity of the targeted condition. The desired purity can be achieved by introducing a sorting step. For uses provided herein, the HSPCs are generally in a volume of a liter or less, can be 500 ml or less, even 250 ml or 100 ml or less. The clinically relevant number of HSPCs can be apportioned into multiple infusions that cumulatively equal or exceed the desired total amount of HSPCs.

### Methods of the Present Invention

Accordingly, the first object of the present invention relates to a method of assessing the exhaustion of a population of hematopoietic stems cells (HSCs) obtained from a subject comprising determining the expression level of one or more genes selected from the group consisting of: IFI44L, STAT2, IRF9, MX1, SAMD9L, CEBPB, BRD7, CD69, EGR1, GBP2, H1FX, HLA-B, HLA-DQA1, ISG15, ISG20, JUND, LAP3, LGLALS3BP, LMO2, LY6E, MLF1, NCOA7, NR4A1, NR4A2, SETBEP1, TAF10, TCF4, TOP2B, TSC22D3, VAMP8, YBX3, ZNF385D, ZSCAN31, ENO1, HIST2H2BE, SREBF1, BAZ2B, BTG2, JUNB, MAFG, NFIB, ZNF439, PARP14, SPEN, MBD2, BTG1, CBX6, EIF2AK2, PSIP1, PURA, SAMD9, CASP8, CD74, HDGF, LYL1, SND1, ZBTB20, MBD3, MX2, ARL4A, DEK, PBXIP1, PQLR2L, TNFSF10, ID3, KLF12, MAF1, TRIL22, FOSL1, OAS1, TOB1, ZNF544, MLLT3, PAWR, and ZNF618, wherein the expression level indicates whether said population of HSCs is exhausted.

In some embodiments, the method comprises determining the expression level of one or more genes selected from the group consisting of IFI44L, STAT2, IRF9, MX1, SAMD9L, and CEBPB wherein the expression level indicates whether said population of HSCs is exhausted.

In some embodiments, the expression level of CEBPB and the expression level of one or more genes selected from the group consisting of IFI44L, STAT2, IRF9, MX1, and SAMD9L are determined.

In some embodiments, the method of the present invention comprised the steps of i) determining the expression level of one or more genes in the population of HSCs ii) comparing the expression level with their corresponding predetermined reference value wherein a difference between the level determined at step i) and the predetermined reference value is indicative whether said population of HSCs is exhausted.

In some embodiments, the method of the present invention comprised the steps of i) determining the expression level of one or more genes in the population of HSCs ii) comparing the expression level with their corresponding predetermined reference value wherein a difference between the level determined at step i) and the predetermined reference value is indicative whether said population of HSCs is exhausted.

Typically, when the expression level is higher than the predetermined reference value, then it is concluded that the population of HSCs is exhausted. On contrary, when the expression level is lower than the predetermined reference value, then it is concluded that the population of HSCs is not exhausted. Practically, high statistical significance values (e.g. low P values) are generally obtained for a range of successive arbitrary quantification values, and not only for a single arbitrary quantification value. Thus, in some embodiments, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above. For example, according to this specific embodiment of a “cut-off” value, the outcome can be determined by comparing the expression level with the range of values which are identified. In some embodiments, a cut-off value thus consists of a range of quantification values, e.g. centred on the quantification value for which the highest statistical significance value is found (e.g. generally the minimum p value which is found). For example, on a hypothetical scale of 1 to 10, if the ideal cut-off value (the value with the highest statistical significance) is 5, a suitable (exemplary) range may be from 4-6. For example, a subject may be assessed by comparing values obtained by determining the expression level of one or more genes as disclosed, where values greater than 5 reveal that the population of HSCs is exhausted and values less than 5 reveal that the population of HSCs is not exhausted. In some embodiments, a subject may be assessed by comparing values obtained by measuring the expression level and comparing the values on a scale, where values above the range of 4-6 indicate that the population of HSCs is exhausted and values below the range of 4-6 indicate that the population of HSCs is not exhausted, with values falling within the range of 4-6 indicating that further explorations have to be carried out for determining whether the population of HSCs is exhausted.

In some embodiments, a score which is a composite of the expression levels of the different biomarkers is determined and compared to the predetermined reference value wherein a difference between said score and said predetermined reference value is indicative whether the population of HSCs is exhausted.

In some embodiments, the method of the invention comprises the use of a classification algorithm typically selected from Linear Discriminant Analysis (LDA), Topological Data Analysis (TDA), Neural Networks, Support Vector Machine (SVM) algorithm and Random Forests algorithm (RF) such as described in the Example. In some embodiments, the method of the invention comprises the step of determining the subject response using a classification algorithm. As used herein, the term “classification algorithm” has its general meaning in the art and refers to classification and regression tree methods and multivariate classification well known in the art such as described in U.S. Pat. No. 8,126,690; WO2008/156617. As used herein, the term “support vector machine (SVM)” is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase. In general, SVMs provide a model for use in classifying each of n subjects to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction (Vapnik, 1998 Statistical Learning Theory. New York: Wiley). Any new observation is then classified as belonging to any one of the categories of interest, based where the observation lies in relation to the hyperplane. When more than two categories are considered, the process is carried out pairwise for all of the categories and those results combined to create a rule to discriminate between all the categories. As used herein, the term “Random Forests algorithm” or “RF” has its general meaning in the art and refers to classification algorithm such as described in U.S. Pat. No. 8,126,690; WO2008/156617. Random Forest is a decision-tree-based classifier that is constructed using an algorithm originally developed by Leo Breiman (Breiman L, “Random forests,” Machine Learning 2001, 45:5-32). The classifier uses a large number of individual decision trees and decides the class by choosing the mode of the classes as determined by the individual trees. The individual trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set. In some embodiments, the score is generated by a computer program.

In some embodiments, the method of the present invention comprises a) determining the expression level of a plurality of genes as described above; b) implementing an algorithm on data comprising the expression levels so as to obtain an algorithm output; c) determining whether the population of HSCs is exhausted from the algorithm output of step b).

The algorithm of the present invention can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

The expression level of one or more genes in the population of HSCs may be determined by any suitable method.

Any reliable method for measuring the expression level of a gene may be used. For instance, mRNA can be detected and quantified from a sample (including fractions thereof), such as samples of isolated RNA by various methods known for mRNA, including, for example, amplification-based methods (e.g., Polymerase Chain Reaction (PCR), Real-Time Polymerase Chain Reaction (RT-PCR), Quantitative Polymerase Chain Reaction (qPCR), rolling circle amplification, etc.), hybridization-based methods (e.g., hybridization arrays (e.g., microarrays), NanoString analysis, Northern Blot analysis, branched DNA (bDNA) signal amplification, in situ hybridization, etc.), and sequencing-based methods (e.g., next-generation sequencing methods, for example, using the Illumina or IonTorrent platforms). Other exemplary techniques include ribonuclease protection assay (RPA) and mass spectroscopy.

Typically, the population of HSCs results from results from a stem cell mobilization.

As used herein, the term “mobilization” or “stem cell mobilization” refers to a process involving the recruitment of stem cells from their tissue or organ of residence to peripheral blood following treatment with a mobilization agent. This process mimics the enhancement of the physiological release of stem cells from tissues or organs in response to stress signals during injury and inflammation. The mechanism of the mobilization process depends on the type of mobilization agent administered. Some mobilization agents act as agonists or antagonists that prevent the attachment of stem cells to cells or tissues of their microenvironment. Other mobilization agents induce the release of proteases that cleave the adhesion molecules or support structures between stem cells and their sites of attachment.

As used herein, the term “mobilization agent” refers to a wide range of molecules that act to enhance the mobilization of stem cells from their tissue or organ of residence, e.g., bone marrow (e.g., CD34+ stem cells) and spleen (e.g., Hox11+ stem cells), into peripheral blood. Mobilization agents include chemotherapeutic drugs, e.g., cyclophosphamide and cisplatin; cytokines, and chemokines, e.g., granulocyte colony-stimulating factor (G-CSF), granulocyte-macrophage colony-stimulating factor (GM-CSF), stem cell factor (SCF), Fms-related tyrosine kinase 3 (flt-3) ligand, stromal cell-derived factor 1 (SDF-1); agonists of the chemokine (C—C motif) receptor 1 (CCR1), such as chemokine (C—C motif) ligand 3 (CCL3, also known as macrophage inflammatory protein-1α (Mip-1α)); agonists of the chemokine (C—X—C motif) receptor 1 (CXCR1) and 2 (CXCR2), such as chemokine (C—X—C motif) ligand 2 (CXCL2) (also known as growth-related oncogene protein-β (Gro-β)), and CXCL8 (also known as interleukin-8 (IL-8)); agonists of CXCR4, such as CTCE-02142, and Met-SDF-1; Very Late Antigen (VLA)-4 inhibitors; antagonists of CXCR4, such as TG-0054, plerixafor (also known as AMD3100), and AMD3465, or any combination of the previous agents. A mobilization agent increases the number of stem cells in peripheral blood, thus allowing for a more accessible source of stem cells for use in transplantation, organ repair or regeneration, or treatment of disease.

In some embodiments, the subject suffers from a genetic blood cell disease. In some embodiments, the subject suffers from a Primary Immune Deficiency such as ADA-Deficient Severe Combined Immune Deficiency, X-linked Severe Combined Immune Deficiency, Wiskott-Aldrich Syndrome, X-linked Chronic Granulomatous Disease, Leukocyte Adhesion Deficiency, Hemophagocytic Lymphohistiocytosis, X-linked Hyper IgM Syndrome, X-linked Lymphoproliferative Disease, X-linked Agammaglobulinemia or Common Variable Immunodeficiency. In some embodiments, the subject suffers from a Hemoglobinopathy such as Sickle Cell Disease or β-thalassemia. In some embodiments, the subject suffers from a Storage and Metabolic Disorder such as Gaucher Disease and other lipidoses, Mucopolysaccharidoses (I-VII), X-linked Adrenoleukodystrophy, Metachromatic Leukodystrophy, or Osteopetrosis. In some embodiments, the subject suffers from a Congenital Cytopenias and Stem Cell Defect, such as Fanconi's Anemia, Schwachman-Diamond Syndrome, or Kostmann's Syndrome.

In some embodiments, the subject suffers from X-linked chronic granulomatous disease.

The method of the present invention is particularly suitable for predicting the engraftment defect of a population of HSCs.

In particular, when it is determined that the population of HSCs is exhausted then it can be decided to postpone the engraftment and to administer the subject with a particular treatment e.g. for restoring self-renewal potential of the HSCs that is critical for maintaining the long-term durability of the graft. In particular, the subject may be administered with an anti-inflammatory drug that will thus limit the inflammatory stress of the HSCs.

As used herein, the term “anti-inflammatory drug” relates to compounds that reduce inflammation. This can be, e.g., steroids, just like specific glucocorticoids (often referred to as corticosteroids), which reduce inflammation or swelling by binding to glucocorticoid receptors. The term further encompasses non-steroidal anti-inflammatory drugs (NSAIDs), which counteract the cyclooxygenase (COX) enzyme. The term further encompasses Immune Selective Anti-Inflammatory Derivatives (ImSAIDs), which are a class of drugs that alter the activation and migration of inflammatory cells, which are immune cells responsible for amplifying the inflammatory response.

In some embodiments, the anti-inflammatory drug is an inhibitor of interferons. As used herein, the term “inhibitor of interferons” refers to any compound that is able to inhibit the activity or expression of interferons. For example, the inhibitor can block the interferon or block the signalling pathway. In particular, the inhibitor inhibits the binding of interferons to their receptor. Typically, the inhibitor include polypeptides, antibodies, and inhibitors of expression. In some embodiments, the inhibitor is a neutralizing antibody. Examples of neutralizing interferon antibodies include but are not limited anifrolimab, sifalimumab, rontalizumab, and AGS-009. In some embodiments, the

In some embodiments, the anti-inflammatory drug is an inhibitor of IL-1. As used herein, the term “inhibitor of IL-1” refers to any compound that is able to inhibit the activity or expression of IL-1. For example, the inhibitor can block IL-1 or block the signalling pathway. In particular, the inhibitor inhibits the binding of IL-1 to its receptor. Typically, the inhibitor include polypeptides, antibodies, and inhibitors of expression. In some embodiments, the inhibitor is a neutralizing antibody. Neutralizing antibodies include but are not limited to Exemplary IL-1 inhibitors are disclosed in the following references: U.S. Pat. Nos. 5,747,444; 5,359,032; 5,608,035; 5,843,905; 5,359,032; 5,866,576; 5,869,660; 5,869,315; 5,872,095; 5,955,480; and 5,965,564; International Patent Application Publication Nos WO98/21957, WO96/09323, WO91/17184, WO96/40907, WO98/32733, WO98/42325, WO98/44940, WO98/47892, WO98/56377, WO99/03837. WO99/06426, WO99/06042, WO91/17249, WO98/32733, WO98/17661, WO97/08174, WO95/34326, WO99/36426, and WO99/36415; European patent applications Publication Nos. EP534978 and EP89479; and French patent application no. FR 2762514. The disclosures of all of the aforementioned references are hereby incorporated by reference. Preferred receptor antagonists (including IL-1ra and variants and derivatives thereof), as well as methods of making and using thereof, are described in U.S. Pat. No. 5,075,222; International Patent Application Publication Nos. WO 91/08285; WO 91/17184; WO92/16221; WO93/21946; WO 94/06457; WO 94/21275; WO 94/21235; DE 4219626, WO 94/20517; WO 96/22793; WO 97/28828; and WO 99/36541, Australian Patent Application No. AU9173636; and French Patent Application No. FR2706772; the disclosures of which are incorporated herein by reference. Recombinant IL-1R antagonist protein for use as an anti-inflammatory drug has been commercialized: Anakinra, sold under the trade name ‘Kinneret’ (See U.S. Pat. No. 5,075,222).

In some embodiments, the anti-inflammatory drug is a JAK/STAT inhibitor. JAK inhibitors are well known in the art. For example, JAK inhibitors include phenylaminopyrimidine compounds (WO2009/029998), substituted tricyclic heteroaryl Is (WO2008/079965), cyclopentyl-propanenitrile compounds (WO2008/157208 and WO2008/157207), indazole derivative compounds (WO2008/114812), substituted ammo-thiophene carboxylic acid amide compounds (WO2008/156726), naphthyridine derivative compounds (WO2008/112217), quinoxaline derivative compounds (WO2008/148867), pyrrolopyrimidine derivative compounds (WO2008/119792), purinone and imidazopyridinone derivative compounds (WO2008/060301), 2,4-pyrimidinediamine derivative compounds (WO2008/118823), deazapurine compounds (WO2007/117494) and tricyclic heteroaryl compounds (WO2008/079521). Examples of JAK inhibitors include compounds disclosed in the following publications: US2004/176601, US2004/038992, US2007/135466, US2004/102455, WO2009/054941, US2007/134259, US2004/265963, US2008/194603, US2007/207995, US2008/260754, US2006/063756, US2008/261973, US2007/142402, US2005/159385, US2006/293361, US2004/205835, WO2008/148867, US2008/207613, US2008/279867, US2004/09799, US2002/055514, US2003/236244, US2004/097504, US2004/147507, US2004/176271, US2006/217379, US2008/092199, US2007/043063, US2008/021013, US2004/152625, WO2008/079521, US2009/186815, US2007/203142, WO2008/144011, US2006/270694 and US2001/044442. JAK inhibitors further include compounds disclosed in the following publications: WO2003/011285, WO2007/145957, WO2008/156726, WO2009/035575, WO2009/054941, and WO2009/075830. JAK inhibitors further include compounds disclosed in the following patent applications: U.S. Ser. Nos. 61/137,475 and 61/134,338.

Specific JAK inhibitors include AG490, AUB-6-96, AZ960, AZD1480, baricitinib (LY3009104, INCB28050), BMS-911543, CEP-701, CMP6, CP352,664, CP690,550, CYT-387, INCB20, Jak2-IA, lestaurtinib (CEP-701), LS104, LY2784544, NS018, pacritinib (SB1518), Pyridone 6, ruxolitinib (INCB018424), SB1518, TG101209, TG101348 (SAR302503), TG101348, tofacitinib (CP-690,550), WHI-PI 54, WP1066, XL019, and XLOI 9. Ruxolitinib (Jakafi™, INCB018424; (3R)-3-cyclopentyl-3-[4-(7H-pyrrolo[2,3-d]pyrimidin-4-yl) pyrazol-1-yl]propanenitrile) is a potent, orally available, selective inhibitor of both JAK1 and JAK2 of the JAK-STAT signaling pathway. CYT387 is an inhibitor of Janus kinases JAK1 and JAK2, acting as an ATP competitor with IC50 values of 11 and 18 nM, respectively. TG101348 (SAR302503) is an orally available inhibitor of Janus kinase 2 (JAK-2). TG101348 acts as a competitive inhibitor of protein kinase JAK-2 with IC50=6 nM; related kinases FLT3 and RET are also sensitive, with IC50=25 nM and IC50=17 nM, respectively. AZD1480 is an orally bioavailable inhibitor of Janus-associated kinase 2 (JAK2) with potential antineoplastic activity. JAK2 inhibitor AZD 1480 inhibits JAK2 activation, leading to the inhibition of the JAK/STAT (signal transducer and activator of transcription) signaling including activation of STAT3. Lestaurtinib (CEP-701) is a tyrosine kinase inhibitor structurally related to staurosporine. Pacritinib (SB 1815) is an orally bioavailable inhibitor of JAK2 and the JAK2 mutant JAK2V617F. Pacritinib competes with JAK2 for ATP binding, which may result in inhibition of JAK2 activation, inhibition of the JAK-STAT signaling pathway, and therefore caspase-dependent apoptosis. Baricitinib (LY3009104, INCB28050) is an orally bioavailable inhibitor of JAK1 and JAK2 with IC50=5.9 nm and IC50-5.7, nm respectively. Baricitinib preferentially inhibits JAK1 and JAK2, with 10-fold selectivity over Tyk2 and 100-fold over JAK3. XL019 is an orally bioavailable inhibitor of Janus-associated kinase 2 (JAK2). XL019 inhibits the activation of JAK2 as well as the mutated form JAK2V617F. NS018 is a potent JAK2 inhibitor with some inhibition of Src-family kinases. NS018 has been shown to be highly active against JAK2 with a 50% inhibition (IC50) of <1 nM, and had 30-50-fold greater selectivity for JAK2 over other JAK-family kinases.

In case wherein, it is considered that the population of HSCs is not exhausted in can then be proceeded directly with the engraftment of the population. Previously to the engraftment the cells may be manipulated for enhancing their therapeutic potential. In particular, the cells are genetically engineered so as to correct a particular gene deficit by expression a particular transgene of interest or by repressing the expression of a particular gene. As used herein, the term “genetically engineered HSC” or “genetically modified HSC” refers to a cell or cells that have undergone gene editing so as to alter a target gene in the cell's genome, or have been altered such that an exogenous gene or exogenous gene sequence is expressed in the cell.

A further object of the present invention relates to a method of therapy in need thereof comprising i) determining whether the population of HSCs from a donor is exhausted by performing the method as above described, and ii) administering a therapeutically effective amount of the population of HSCs in a recipient when it is concluded that the population is not exhausted.

In some embodiments, the method of the present invention comprises the step of full ablating hematopoiesis in the recipient patient before administering the therapeutically effective amount of the population of HSCs. Preparative or conditioning regimens are well known in the art and typically include chemotherapy-based regimens.

In some embodiments, the method of the present invention comprises the step of genetically engineering the population of HSCs before administering the therapeutically effective amount of the population of HSCs.

The invention will be further illustrated by the following figures and examples. However, these examples and figures should not be interpreted in any way as limiting the scope of the present invention.

### FIGURES

FIG. 1. HSCs with an altered state and aberrant CEBPB expression. Boxplots of CEBPB mRNA expression in the HSC, HSC-enriched, MPP, NeutroP0, NeutroP1, NeutroP2 and NeutroP3 populations, in each individual.

FIG. 2. Expression levels of biomarkers in CGD HSCs that correlate with poor engraftment after GT. Boxplots of the expression by HSCs of 10 ISGs (IFI44L, LGALS3BP, LY6E, EIF2AK2, STAT2, ISG15, IRF9, MX1, MX2, and SAMD9L gene) in each individual, identified by the elastic-net model as being predictive of the poor engraftment in P2 and P5.

FIG. 3. Xenotransplantation of the patients' corrected HSPCs in a humanized mouse model

HSPCs from P4 and P5's IMPs were infused into NOD-SCID-γc−/− (NSG) mice (n=4 per group, VCN_{P4}=1.7, VCN_{P5}=1.6). Nontransduced CB samples (n=3) and transduced MPB samples (n=5, VCNMPB=3.47) were used as controls, with the same (clinical) ex vivo cell engineering protocol. Engraftments in the BM and spleen were analyzed 16 weeks after transplantation.
(A) Left, human chimerism (% hCD45⁺/(hCD45⁺+mCD45⁺)) in the BM 16 weeks after transplantation (P4 n=4, P5 n=4, CB n=3, MPB n=5). The level of chimerism was significantly lower in P5 than in P4 (p=0.0286 in a Mann-Whitney test). Right, the number of human CD45+ cells in the BM. ns: no significant.
(B) Frequency of human CD45+CD34+ cells in the BM. The frequency was significantly lower in P5 than in P4 (p=0.0286 in a Mann-Whitney test).
(C) Left, human chimerism (% hCD45⁺/(hCD45⁺+mCD45⁺)) in the spleen 16 weeks after transplantation, in the same mice. Right, the number of human CD45+ cells in the spleen. The level of chimerism was significantly lower in P5 than in P4 (p=0.0286 in a Mann-Whitney test).
(D) The VCN per cell was measured to assess the level of gene marking in total BM, using a droplet digital PCR (ddPCR) technique. The VCN was significantly lower in P5 than in P4 (p=0.0286 in a Mann-Whitney test).
(E) The VCN per cell was measured to assess the level of gene marking in the total spleen, using a ddPCR technique. The VCN was significantly lower in P5 than in P4 (p=0.0286 in a Mann-Whitney test).
(F) The frequency of Gp91+ cells in human CD45+BM. The frequency was significantly lower in P5 than in P4 (p=0.0286 in a Mann-Whitney test).

### EXAMPLE

### Introduction:

Chronic granulomatous disease (CGD) is a recessive inborn error of immunity (Bousfiha et al., 2020; Tangye et al., 2020) caused by loss-of-function (LOF) mutations in the X-linked or autosomal genes that encode the five components of the NADPH oxidase complex (D. Roos et al., 2017).

The complex's membrane-bound catalytic core is a heterodimer of gp91phox and p22phox, encoded respectively by the X-linked CYBB gene and the autosomal CYBA gene. The regulatory part is a cytosolic heterotrimer composed of p40phox, p47phox and p27phox, encoded respectively by NCF4, NCF1, and NCF2 (Di. Roos, 2016). Following phagocyte activation, the NADPH oxidase complex assembles on the phagosomal membrane and produces reactive oxygen species that can destroy microorganisms.

Patients with CGD suffer from specific, recurrent, invasive, life-threatening bacterial and fungal infections (Marciano et al., 2015; van den Berg et al., 2009). Prominent inflammatory manifestations (particularly affecting the respiratory and gastrointestinal tracts) are also common-especially in patients with the X-linked form of the disease (Magnani et al., 2014; Marciano et al., 2017). In some patients, CGD is revealed by these inflammatory manifestations. Others present initially with unexplained granulomatosis, which is associated with a poor prognosis (Marciano et al., 2017; van de Veerdonk & Dinauer, 2017).

Patients routinely receive antimicrobial prophylaxis, and the only widely available, curative treatment is allogeneic hematopoietic stem cell transplantation (HSCT) (Chiesa et al., 2020; Cole et al., 2013; Gennery et al., 2010; Martinez et al., 2012). After conditioning, CD34+ hematopoietic stem and progenitor cells (HSPCs) are transplanted; engraftment of the most immature hematopoietic stem cells (HSCs) in the bone marrow (BM) then enables full immune reconstitution. In the absence of a compatible donor for HSCT, gene therapy (GT) is a treatment option. Although several research groups have developed GT protocols for CGD, the prior clinical trials have been compromised by the absence of stable engraftment of gene-corrected cells (Grez et al., 2011). In the first trials (without a conditioning regimen), the lack of engraftment was probably due to the absence of a selective advantage for the transduced cells (Grez et al., 2011; Malech et al., 1997). The use of a reduced conditioning regimen in the subsequent trials with gammaretroviral vectors resulted in temporary engraftment, although insertional mutagenesis favored the development of myelodysplastic syndromes in a few patients (Ott et al., 2006; Stein et al., 2010). More recently, significant improvements were achieved after the gammaretroviral vectors had been replaced by self-inactivating lentiviral vectors in which a chimeric internal promoter drove gp91phox expression specifically in myeloid cells (Brendel et al., 2018). The combination of this new-generation vector with a full busulfan-based myeloablative conditioning regimen resulted in significantly better clinical and biological outcomes and a better safety profile (i.e. the absence of GT-related adverse events treatment) in two trials (a trial in London sponsored by Généthon (ClinicalTrials.gov identifier: NCT01855685) and Kohn et al.'s investigator-led trial in the USA (NCT02234934)). In 9 of the 13 treated patients, the stably engrafted cells cured the underlying CGD (Kohn et al., 2020).

Several recent studies have reported that chronic inflammation harms HSPCs in patients with CGD and patients with other conditions. Mice and humans with X-CGD have low HSC counts in the BM (Weisser et al., 2016). Furthermore, human HSCs from patients with CGD showed rapid exhaustion after in vitro culture. In the presence of high levels of pro-inflammatory cytokines (such as IL-1β), mouse HSCs showed increased cycling and a lower long-term engraftment potential (Weisser et al., 2016). Elevated levels of IL-18 and IFNγ have been observed in inflamed tissue from patients with CGD (Meda Spaccamela et al., 2019).

Here, we report the results of a Phase I/II clinical trial of GT (based on a G1XCGD lentiviral vector and gene-modified HSPCs) in four patients with X-CGD lacking an human leukocyte antigen (HLA)-compatible donor for HSCT. A fifth patient was included in the clinical trial, but not treated because the investigational medicinal product (IMP) did not meet the release criteria. The degrees of cell engraftment and clinical efficacy varied markedly from one patient to another. In order to fully understand the molecular alterations in HSCs associated with the success or failure of GT for CGD, we profiled the transcriptome of HSPCs at the single-cell level and found that the engraftment defect was correlated with the upregulation of interferon pathways and a set of predictive biomarkers.

### Methods

### Patients

All the patients were treated in the Pediatric Immunohematology Department or the Adult Hematology Department at Necker Children's Hospital (Paris, France). The IMP was manufactured in the hospital's Cell and Gene Therapy Laboratory and Biotherapy Department. The follow-up included regular hospital consultations and laboratory tests (including immune cell hematological reconstitution, gene marking in cell subpopulations (VCN analyses), gp91phox expression, and the DHR oxidative burst assay used to assess the activity of NADPH oxidase. Additional cell characterization assays were performed on an ad hoc basis.

### Healthy Donors

Mobilized peripheral blood (MPB) samples were provided by HemaCare (Northridge, CA, USA). CD34+ cells were mobilized with G-CSF and plerixafor (for HD1-2) or with plerixafor only (for HD3-4). HD5-7 were mobilized with G-CSF, and the CD34+ cells were harvested and separated in the Department of Biotherapy at Necker Children's Hospital. The HDs provided written, informed consent to the use of their samples for research purposes, and their data were anonymized. No nominative data concerning the donor were sent to the investigators. Cord blood was obtained from a biological resources center (Centre Ressources Biologiques (CRB))—Banque de Sang de Cordon) at Saint-Louis Hospital (Paris, France). HSPCs were isolated using standard Ficoll density gradient centrifugation and then magnetic selection on a column with anti-CD34+ antibody.

Blood samples from HD8-12 were obtained from the French Blood Establishment (Etablissement Français du Sang, Paris, France; reference: C CPSL UNT-No18/EFS/032). Again, the HDs provided written, informed consent to the anonymous use of their samples for research purposes. PBMCs were isolated using standard Ficoll density gradient centrifugation.

### Determination of the VCN

Genomic DNA was extracted from HSPCs in the IMP (14 days after transduction) and during the follow-up from sorted neutrophils, monocytes, T cells, B cells and NK cells and on total PBMCs using a DNeasy Kit (Qiagen).

The VCN was determined in a quantitative PCR assay (Viia 7, Applied Biosystems) and the PSI and ALB human probes (see Key Resource table).

### The DHR Assay

Neutrophils were stimulated with phorbol myristate acetate to induce superoxide anion production. The non-fluorescent dye DHR is reduced by H₂O₂ and thus converted into fluorescent rhodamine, which is quantified using flow cytometry.

### Isolation of Mononuclear Cells

In line with the trial protocol, peripheral blood was sampled regularly during the follow-up period, whereas MPB was sampled once for GT. Mononuclear cells were isolated from PB or MPB using standard Ficoll density gradient separation. The absolute lymphocyte count was determined using Trucount Tubes (BD Bioscience).

### Flow Cytometry

The neutrophil subpopulation was purified from PB on a column with magnetic beads and fluorochrome-coupled anti-CD15 antibodies. Monocytes, T cells, B cells and NK cells were sorted on a cell sorter (FACSAria II, BD Biosciences), using fluorochrome-coupled antibodies against CD14, CD3, CD19, and CD56.

Total PBMCs were surface-stained for Gp91, using an anti-flavocytochrome b558 7D5 clone (human) mAb-FITC (MBL Bio) and gating for neutrophils.

The patients' HSPCs were characterized using a multilabeled panel with the following antibodies: CD34 (clone 581, Beckman Coulter), lineage cocktail: CD2, CD3, CD4, CD8, CD14, CD15, CD16, CD19, CD20, CD33, CD56, CD235a (BD Biosciences), CD133 (clone 293C3, Miltenyi Biotec), CD38 (clone HIT2, BD Biosciences), CD90 (clone 5E10, BD Biosciences), and CD45RA (clone T6D11, Miltenyi Biotec). Staining was analyzed with a FACSCanto II cell analyzer.

### Analysis of Vector Integration Sites

Integration site were identified using the S-EPTS/LM-PCR protocol, an advanced version of EPTS/LM-PCR (Schmidt et al., 2001), and thereafter analyzed using the GENE-IS tool suite (Afzal, Wilkening, et al., 2017).

### Bulk RNA-Seq

RNA was isolated using RNeasy Micro Kit (Qiagen) with a DNase step. RNA integrity and concentration were assessed using capillary electrophoresis and the Fragment Analyzer (Agilent). RNA-seq libraries were prepared from 100 ng of total RNA, using the Universal Plus mRNA stranded (Nugen-Tecan). The amplified cDNA produced was sequenced on a NovaSeq6000 system (Illumina). There were ˜50 million reads per library.

The raw read counts were normalized with DESeq2 package, based on the library size and testing for differential expression between conditions (Love et al., 2014). Coding genes were extracted from gencodeV30, then noise filter with any gene expression more than 20 were applied before the pathway enrichment analysis. Normalized enrichment scores were calculated for all deregulated coding genes, using GSEA software (Subramanian et al., 2005).

Gene set enrichment was investigated with MSigDB, using an hypergeometric test on a pre-filter dataset (p<0.05 and fold-change (FC)>1.2 or <−1.2). The output false discovery rate had to be below 0.05.

Representation and quantification of module activity (ROMA) was applied to DEGs in PBMCs and HSPCs. ROMA calculate a module score for a set of samples and is based on the simplest single-factor linear model of gene regulation whose first principal component approximates the expression data (Martignetti et al., 2016).

### Single-HSPC RNA-Seq

### Library Preparation

Frozen HSPCs from each individual were thawed and resuspended in PBS+1% BSA. The cell preparation was loaded onto a Chromium Single-Cell Chip (10× Genomics) for co-encapsulation with barcoded Gel Beads at a target capture rate of ˜7000 individual cells per sample. Captured mRNAs were barcoded during cDNA synthesis, using the Chromium Single-Cell 3 reagents v3 (10× Genomics) according to the manufacturer's instructions. All samples were processed simultaneously with the Chromium Controller (10× Genomics), and the resulting libraries were prepared in parallel in a single batch. We pooled all the libraries for sequencing in a single SP Illumina flow cell. Libraries were sequenced with 28 read 1 cycles containing cell-identifying barcodes and unique molecular identifiers (UMIs), 8 i7 index cycles, and 91 read 2 cycles containing transcript sequences on an Illumina NovaSeq 6000 (Illumina). Sequencing reads were demultiplexed and aligned with the human reference genome (GRCh38), using the CellRanger Pipeline v3.1.

### Integration and Data Pre-Processing

Empty droplets were excluded with the DropletUtils package. Cells with more of 15% of mitochondrial genes and less than 3000 UMIs were removed. Cells expressing more than 50 genes and 6000 high variable genes were selected using Seurat.

### Akira:

Empty droplets were excluded with DropletUtils package with an FDR threshold of 0.01. Cells with more than 15% of mitochondrial genes and less than 3000 UMI were removed. As HSPC presents different cell maturity which translates to variable expression abundance across cells, the expression matrix normalization for each sample was conducted using scran normalization by deconvolution methods in place of standard library size normalization (Lun et al., 2016). The gene expression was then restricted to only the protein coding genes and then selecting the 6000 high variables genes found with Seurat FindVariableFeatures function with the default parameter.

### CellID Annotation of Individual Cells, Using HSPC Reference Signatures

CellID is a robust statistical method for gene signature extraction and cell identity recognition on the basis of single-cell RNA-seq data (Cortal et al., 2021). It incorporates a multiple correspondence analysis and simultaneously represents cells and genes in low-dimension space. The genes are then ranked by their Euclidean distance from each individual cell, which provides unbiased per-cell gene signatures.

Using published data (Velten et al., 2017), CellID, and the 200 most specific genes, we extracted 16 reference signatures: HSC, MPP, MLP, ImP1, ImP2 (corresponding to common myeloid progenitors), NeutroP0, NeutroP1, NeutroP2, NeutroP3, MonoDCP (corresponding to granulocyte-monocyte progenitors), BcellP, MEP1, MEP2, EryP, MkP, and EoBasMastP. The CellID method defines the gene ranking in each cell in the dataset (53,412 cells in total), evaluates whether a cell accurately matches a particular reference signature, and determines the cell's identity on the basis of the top p-value (p<0.01). The enrichment score is based on the −log 10(p-value).

### HSC Identification and the Diffusion Map

Since the annotated population was enriched in HSCs (corresponding to 30% of all HSPCs), we combined diffusion map analysis (for determining the differentiation trajectory (with an analysis of the enrichment strength for Velten et al.'s HSC signature (to determine the origin of the diffusion map and isolate the most immature HSC subpopulation, corresponding to 3% of the total HSPC.

The other annotated HSCs are referred to as “HSC-enriched”. The “All HSC” subpopulation includes the most immature HSCs and the HSC-enriched subpopulations.

Following cell-type annotation, samples were integrated by applying the Harmony package using the first 30 principal component axis and the default parameter as input.

### The CellID Score for Signaling Pathway Enrichment

The CellID method was used to assess the statistical enrichment of individual-cell gene signatures against signaling pathway gene sets (such as Hallmark gene sets) based on hypergeometric test p-values with Benjamini-Hochberg correction on the number of tested gene signatures. Enrichment score were calculated as the −log 10(p-value) of such test. A cell was considered enriched in a given pathway if the score is >2 (p<0.01).

### CellID Identification of Mixed Signatures, and UpSet Plots

To further understand the heterogeneity and diversity of cell state among the cells, we took advantage of CellID enrichment system to identify cells that were significantly enriched (p<0.01) for several reference signatures.

UpSet plot with UpSetR package for all labels or on selected labels such as NeutroP0, BcellP, MonoDCP, MPP, All HSC and others.

Cells that significantly match (p<0.01) mixed signatures were represented on an UpSet plot with the UpSetR package for all labels or as NeutroP0, BcellP, MonoDCP, MPP, All HSC and Other on selected labels.

### Deregulated Gene Analysis in a MAST

DEGs in HSC subpopulations were identified in a MAST (Finak et al., 2015) from the 6000 highly variable genes in the datasets. To analyzed the enrichment pathway, we applied a hypergeometric test with MSigDB.

### Elastic-Net Logistic Regression for the Identification of Predictive Markers

We used an elastic-net logistic regression model (Couckuyt et al., 2022; Torang et al., 2019; Zou & Hastie, 2005) to predict each cell's ability to engraft or not. We used the glmnet package to test the 239 IFNα/IFNγ genes and transcription factors with differential expression in at least one patient) and determine their contribution to engraftment success (True) or failure (False).

We performed cross-validation using the caret package to determine the optimal lambda (XX) and alpha (0.7) parameters. The model was trained on 75% of the 469 HSCs, comprising 304 true cells (from P1 and P4) and 165 HSC false cells (from P2 and P5). Then we performed 50 models with these parameters and with a random split to check the stability of the results. We obtained AUC between x&x and accuracy between x&x. Using those models, 78 significantly contributing factors were selected by the model as engraftment predictors. We then concentrated on the 51 factors with a negative estimate (i.e. corresponding to detrimental factors for engraftment that were upregulated in P2 and P5). The network of genes selected by the elastic-net model as being detrimental for engraftment was visualized using StringDB.

### Mouse Experiments and Xenotranplantation Assays

All animal procedures were approved by the animal care and use committee at the University of Paris (Paris, France; Feb. 16, 2021) and the French Ministry of Agriculture (APAFIS #29592-2020120216106476). The procedures were performed in accordance with European Union (EU) Directive 2010/63/EU. NOD-SCID-γc^{-/-} strain (NSG) mice were obtained from Charles River Laboratories. 3.5 to 4.2×10⁵ engineered HSPCs from a patient's IMP were injected into 16 NSG mice previously conditioned with one dose per day of busulfan at 15 mg/kg (45 mg/kg in total). Engraftment in BM, spleen and thymus were analyzed after 16 weeks, using flow cytometry. The antibodies used are described in the Key Resource Table. Culture conditions for the MPB control were the same as in the clinical trial: 18 h of pre-activation in a cytokine cocktail (SCF: 300 ng/ml, FLT3L: 300 ng/ml, TPO: 100 ng/ml, IL3: 20 ng/ml), addition of 10 uM PGE2 2 h before transduction, and then 2 rounds of transduction with the Gp91phox clinical vector and. The VCN in the MPB control sample (3.47) was measured in a ddPCR assay (see below). The cells from the CB control were not transduced or cultured.

The VCN in the mice was assessed by ddPCR assay on a Bio-rad QX200 ddPCR System. Quantitative PCR on droplets was performed using TaqMan PCR Master Mix for probes in a Applied Biosystems SimpliAmp thermocycler, using a standard protocol. 80 ng of total gDNA, 900 nM primers and 250 nM probes were used in a total volume of 17 ul for absolute quantification with the droplet reader.

### Statistical Analysis

Analyses of the data distribution and intergroup differences were performed with GraphPad Prism software (version 9) or R Studio software (version 4.0.4).

### Results

### Clinical Presentation of Patients

We performed a non-randomized, open-label, phase I/II clinical study (ClinicalTrials.gov identifier: NCT02757911) of five patients with X-CGD (referred to hereafter as P1 to P5) who received autologous CD34+ cells transduced with a lentiviral GT vector and then cryopreserved. The patients had a severe deficiency in gp91^{phox} protein, due to a mutation in CYBB gene and the absence of NADPH oxidase activity. One patient (P3) was not treated because the level of CD34+ cell transduction did not meet our minimum requirement. The patients' age at the time of GT ranged from 8 to 28 years.

All four treated patients had severe X-CGD-related infections, some of which were active at the time of GT. Three of the patients (P1, P2, and P5) also had severe inflammatory manifestations (Table 1). Prior to GT, all four patients received standard antimicrobial/antifungal prophylaxis and (for those with inflammatory features) long-term treatment with anti-inflammatories.

In particular, P1 (8 years old at the time of GT) presented with many deep abscesses, gut inflammation, and severe lung disease with infectious and inflammatory components; hence, P1 was receiving oxygen therapy and enteral nutrition in addition to steroids and antimicrobial prophylaxis.

P2 and P5 (respectively 19 and 28 years old at the time of GT) had a similar clinical profile, with very severe, long-lasting, corticoresistant episodes of inflammation and typical CGD-associated infections. Since infancy, P2 had presented with treatment-resistant granulomatous cystitis. He also had a history of tibia osteomyelitis and actinomycotic abscesses of the liver with portal hypertension, which had required surgery. P5 presented with long-lasting episodes of severe colitis that were refractory to various anti-inflammatory treatments, together with pulmonary aspergillosis, osteitis, and Campylobacter and Salmonella infections.

In contrast to the other patients, P4 (23 years old at the time of GT) did not have a history of inflammation but presented with extremely severe, invasive, treatment-resistant pulmonary aspergillosis, Salmonella infections, cervical adenitis, and folliculitis. In view of P4's critical condition and the absence of other treatment options, compassionate-use GT was authorized after concomitant modifications in the gene correction process (see below).

### Manufacturing and Characteristics of the IMP

Investigational medicinal products were manufactured from HSPCs harvested from the BM or via leukapheresis after G-CSF/plerixafor cell mobilization. The gene-corrected cells were infused after targeted myeloablative conditioning (median (range) area under the curve for total exposure to busulfan: 75610 (71973-85478) ng/ml·h). The infused CD34+ cell doses ranged from 3.0 to 15.67×10⁶/kg.

P1 received an IMP containing genetically modified CD34+ HSPCs sourced from BM and mobilized peripheral blood (MPB) (G-CSF+Plerixafor-mobilized leukapheresis), as specified in the initial protocol. For P2, a low yield of CD34+ cells after BM harvest prevented gene correction, and so the unmodified cell product was cryopreserved. After two subsequent apheresis with below-specification levels of gene transduction, the transduction protocol was modified with the addition of prostaglandin E2 (PGE2), described to favor HSC transduction and repopulation ability (Zonari et al., 2017). Addition of a transduction adjuvant PGE2 considerably improved the level of lentiviral transduction. The following three procedures (for P4, P2 and P5) were therefore performed with the optimized protocol, starting from G-CSF+Plerixafor-(P4, 5) or Plerixafor (P2)-mobilized leukapheresis. Using classical HSPC phenotyping, we showed that patients had received similar doses of HSCs (defined as 34+Lin−CD38−CD133+CD90+CD45RA− cells) per kg.

Indeed, the addition of PGE2 during the transduction step was associated with a significantly greater vector copy number (VCN) in preclinical test material and in the IMP (p=0.0022 and p=0.0357, respectively) (data not shown). Moreover, transcriptomic analysis of PGE2-treated and control HSPCs from P2 and P4 showed that the addition of this adjuvant was associated with a less inflammatory expression profile (data not shown). For the four treated patients, the median (range) VCN in the IMP was 1.42 (0.99-1.73).

### Clinical Outcomes

After myeloablative-conditioning regimen, patients were infused with the IMP containing genetically modified HSPCs. The infusion was well tolerated in all cases. The only adverse events were related to the conditioning (e.g. mucositis), rather than the infusion. P1 presented with Staphylococcus epidermidis sepsis 6 days after infusion of the IMP, and P2 presented with cytolysis, and cholestasis 14 days after infusion. These adverse events resolved after engraftment, and hematopoietic reconstitution was satisfactory for all patients.

As of January 2022, the median (range) follow-up period was 42 months (24-60). For all treated patients, the VCN in neutrophils ranged from 0.17 to 0.96 in the first month post-GT. In P2 and P5, a progressive decrease in the engraftment of gene-corrected cells was observed 2 to 3 months after GT, and the patients regressed to their pre-GT condition (data not shown). Similar results were observed for monocytes, B cells, NK cells and T cells. The level of gene marking was lower in Tcells, given the absence of T cell depletion during the conditioning (data not shown). Due to the recurrence of inflammation and infections, P2 underwent HSCT with an unrelated, partially matched donor (1 out of 10 HLA alleles was mismatched) 3.5 years after GT. Twenty-seven days after the HSCT, P2 developed ultimately fatal septic shock, in persistent pancytopenia. P1 showed an initial decrease in the level of gene marking, which stabilized at around 10-15% after a few months (data not shown). Although this level was not optimal, it provided P1 with clinical benefit-particularly with regard to the regression of infectious manifestations and as shown by the post-GT lung scan results (data not shown); this enabled P1 to discontinue nocturnal oxygen therapy, enteral nutrition, steroids, and antimicrobial prophylaxis. However, the inflammatory manifestations continue to worsen (particularly in the gut and lung), requiring the recent introduction of Janus kinase (JAK) 1 and 2 inhibitors.

The gene marking results were correlated with the results of the functional oxidative burst (dihydrorhodamine-123 (DHR)) assay in neutrophils, which stabilized at 27% and 47% of neutrophils, for P1 and P4, respectively (data not shown). At last follow-up, the expression of gp91phox protein in CD15+neutrophils was significantly enhanced for P1 and P4 (data not shown).

P4's life-threatening lung aspergillosis resolved completely, with ad integrum healing of the lytic costal erosions as early as 4 months post-GT (data not shown). This stable, clinical benefit was associated with the presence of functional circulating neutrophils (50% of the normal count) (data not shown). P4 resumed his education and is now working full-time. He discontinued all treatments two months post-GT. Seven months after infusion of the IMP, P5 presented with submandibular lymphadenopathy that resolved progressively with oral antibiotic treatment. The patient continued the antimicrobial prophylaxis, and his clinical condition is stable. The analysis of vector integration over time in these patients highlighted the polyclonal reconstitution of both peripheral blood mononuclear cells (PBMCs) and neutrophils (data not shown). The mean (range) number of unique integration sites at last follow-up was 3374 (119-10650) in PBMCs and 4492 (158-15344) in neutrophils. Lower values were observed for P2 and P5, due to the progressive loss of gene-corrected cells. Integrations close to oncogenes (such as MECOM (MDS/EVI1)) previously targeted by gammaretroviral vectors were present at a low frequency (below 2%) in all patients and did not increase over time.

### A Low Frequency of HSCs and a High Frequency of Myeloid Progenitors

To understand the interindividual differences in engraftment, we analyzed transcriptomic differences in PBMCs and HSPCs from patients vs. healthy donors (HDs). This analysis highlighted the upregulation of the type 1 and 2 interferons (IFN) response pathway in PBMCs and HSPCs (data not shown). We also used the ROMA method (Martignetti et al., 2016) to quantify the activity of sets of genes in individual samples. This analysis did not reveal any differences in the patients' PBMCs vs. HD PBMCs. However, P2 and P5's HSPCs had a higher interferon alpha score, a higher interferon gamma score, and a more intense inflammatory response, relative to HDs. In contrast, P4 (the patient with the best engraftment of gene-corrected cells) displayed only a slightly higher interferon alpha score (data not shown). To further explore these interindividual differences in HSPC subpopulations, we performed single-cell transcriptomic analyses and then used the CellID software (Cortal et al., 2021) and 16 different BM HSPC reference signatures (data not shown) (Velten et al., 2017) to determine the transcriptional profiles of 53412 MPB HSPCs from the four treated patients with CGD and four HDs (data not shown). CellID is a robust statistical method developed for gene signature extraction and cell recognition, on the basis of single-cell RNA-seq data.

In order to define the most immature HSC subpopulation, we used a diffusion mapping approach to determine the origin in the trajectory map (data not shown). We then compared the frequency of each subpopulations in the patients vs. HDs. We showed that CGD patients are expected to have around 2-fold less HSC compared with HDs (odds ratio: 0.53; p=2.2×10″ 16, using a logistic regression). Importantly, patients with engraftment failure (P2 and P5) have even a lower HSC proportion than patients with successful engraftment (P1 and P4) (OR=0.58, p=3.78×10⁻⁸, using a logistic regression). Moreover, P2 presented high frequency of B cell progenitors and monocyte/dendritic cell progenitors. P5 had a high neutrophil progenitor (NeutroP0) proportion, and low immature progenitor (ImP1, similar to common myeloid progenitors) proportion (data not shown). No significant differences in frequencies in the various HSPC cell-type were observed for the other two patients (P1 and P4) in comparison with HDs (data not shown).

An Aberrant HSC Profile, with a Mixture of HSC/Neutrophil Signatures

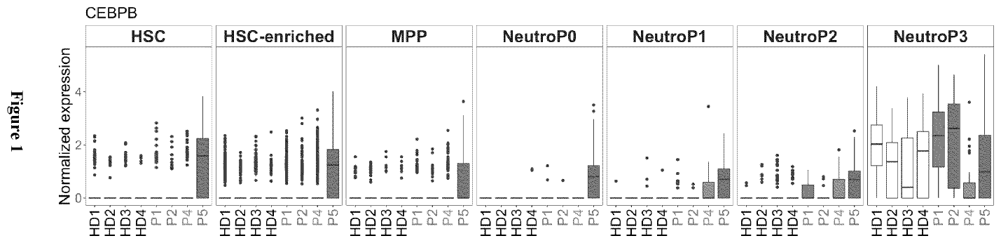

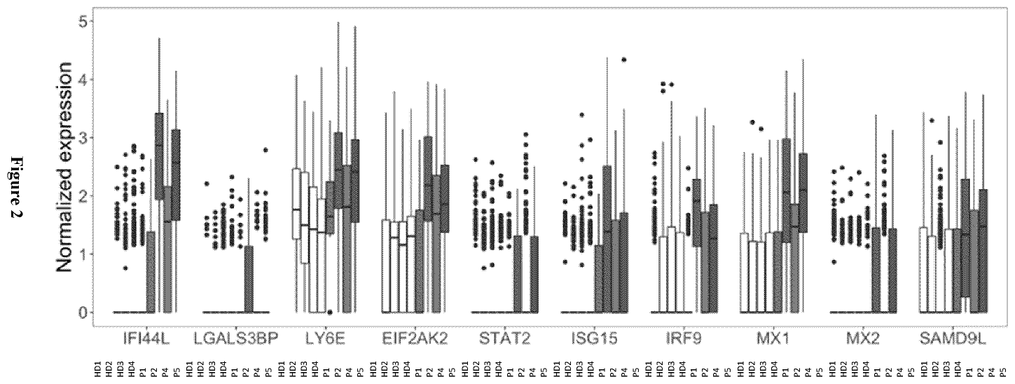

In order to further explore the abnormally large NeutroP0 subpopulation in P5, we used the CellID method to identify cells that simultaneously matched multiple cell-type signatures. Thus, by testing each individual cell against the 16 reference signatures, each individual cell may be identified as simultaneously displaying signatures from several cell types (data not shown). For example, the NeutroP0^{Match} population (data not shown) encompassed not only the NeutroP0^{ID} population (data not shown) but also cells displaying other cell types as their top signatures, yet showing significant enrichments for the NeutroP0 gene signature. An UpSet plot of the various mixed signatures showed that there were 20 distinct combinations of the NeutroP0 signature with other cell types in P5 but only three distinct combinations in HDs (data not shown). We therefore looked further at the most frequent combinations in P5, which comprised NeutroP0 signatures) (data not shown). This analysis revealed that 438 cells matched the NeutroP0, MPP and All HSC signatures. This mixed signature (depicted in black in the Uniform Manifold Approximation and Projection (UMAP) plot (data not shown)) was found in P5 but not in the other patients or the HDs. In this pathological subpopulation, the top genes, defined using CellID, included the CEBPβ transcription factor (data not shown), which is typically expressed more in committed myeloid progenitors. In HD cells, CEBPβ was expressed from the NeutroP3 stage onwards. In P5's cell, CEBPβ was expressed in the most immature HSC subpopulation and in early HSC progenitors and the MPP population (FIG. 1). These results suggest strongly that P5 not only presents a large NeutroP0 population but also has a strong alteration in the most immature HSCs state, with aberrant expression of the CEBPB myeloid factor.

In P2, the CellID analysis highlighted a large number of cells with mixed BcellP-MonoDCP signatures (data not shown). This mixed signature was also detected (albeit to a lesser extent) in HDs (especially HD2) and in the other patients (data not shown). The gene signature detected through CellID in this mixed BcellP-MonoDCP population evidenced the expression of genes known to have a role in B and dendritic cell lineages (e.g. IRF8, SPIB, TLR7, and TNFRSF17) and that were not detected in the equivalent population of HDs (data not shown). These results emphasized the closeness of the relationship between the BcellP and MonoDCP lineages, both of which were unusually prominent in P2.

The Interferon Pathway Score Highlights HSC Alterations Correlated with Poor Engraftment

In order to better understand the molecular changes in the most immature HSCs, we used the model-based analysis of single-cell transcriptomics (MAST) method to define differential expressed genes (DEGs) in patients vs. HDs (Finak et al., 2015). We identified 369 DEGs in the most immature HSC subpopulation and then tested for functional enrichment using the Molecular Signature Database (Liberzon et al., 2015). The IFNα, IFNγ and TNFα pathways were more prominent in patients with CGD than in HDs (data not shown).

CellID allowed us to compare individual cell signatures with well-defined gene sets (such as the Hallmarks collection) associated with particular biological states or processes (Liberzon et al., 2015). The highest interferon gamma response score was found for P2 and P5, especially in most immature HSCs (data not shown). In contrast, HSCs in P1 and P4 (the patients with the best correction and engraftment) had a low interferon gamma response score, which was similar to that found in HDs.

To further understand these interindividual differences and DEGs, we performed a MAST analysis for each individual patient's HSCs. Sixty-one of the DEGs were part of the interferon gamma pathway (data not shown). The degree of deregulation was higher in P2 and P5 than in P1 and P4. In order to determine which interferon gamma pathway and transcription factor genes might contribute significantly to engraftment failure, we took advantage of the large number of samples provided by the single-cell RNA-seq transcriptomic profiling with 469 individual HSCs. Furthermore, we made use of a machine learning approach (elastic-net logistic regression) to predict graft ability for each individual cell (Torang et al., 2019). This model identified a set of 51 interferon genes and transcription factors as being predictive of the engraftment defect in P2 and P5 (area under the curve: >0.9, FIG. S10D-E). The interferon-stimulated genes (ISGs) included IFI44L, MX1, STAT2, IRF9 and SAMD9L, all of which were significantly upregulated in P2 and P5's HSC subpopulation (FIG. 2). In P1 and P4 (patients with successful engraftment), these genes were expressed to the same extent as in HDs or only slightly more. The model also selected predictive transcription factors, which interacted in a functional protein association network (data not shown) linking CEBPB (already identified in P5) with other factors, such as JUND, SREBF1 and MAFG.

Taken as a whole, these transcriptomic data identified specific biomarkers in CGD HSCs. Elevated inflammatory pathway activity was predictive of poor engraftment.

HSC Exhaustion, Revealed by the Impaired Xenotransplantation of HSPCs from Patients with Severe CGD

In order to further understand the changes in HSCs associated with defective engraftment in patients with severe CGD, we evaluated xenotransplantation in a humanized NOD-SCID-γc^{-/-} (NSG) mouse model. The transplanted HSPCs came from P4 and P5, whose IMPs were similar.

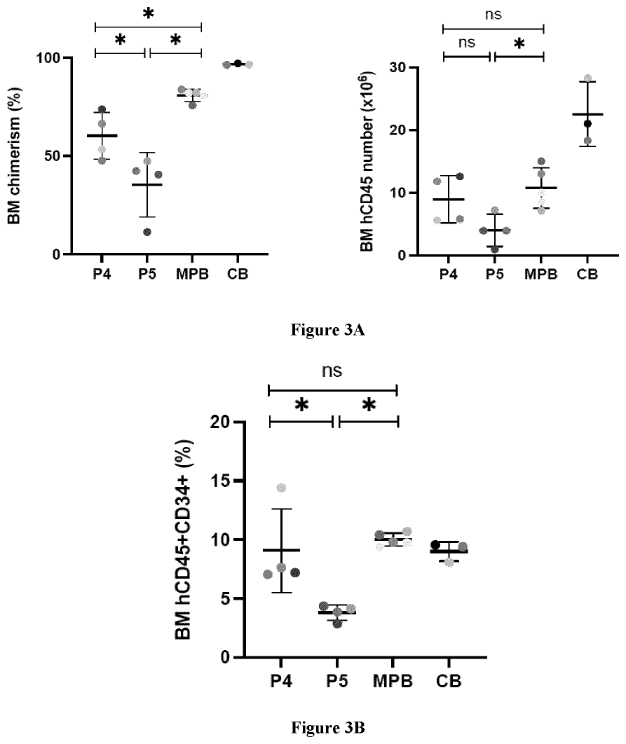

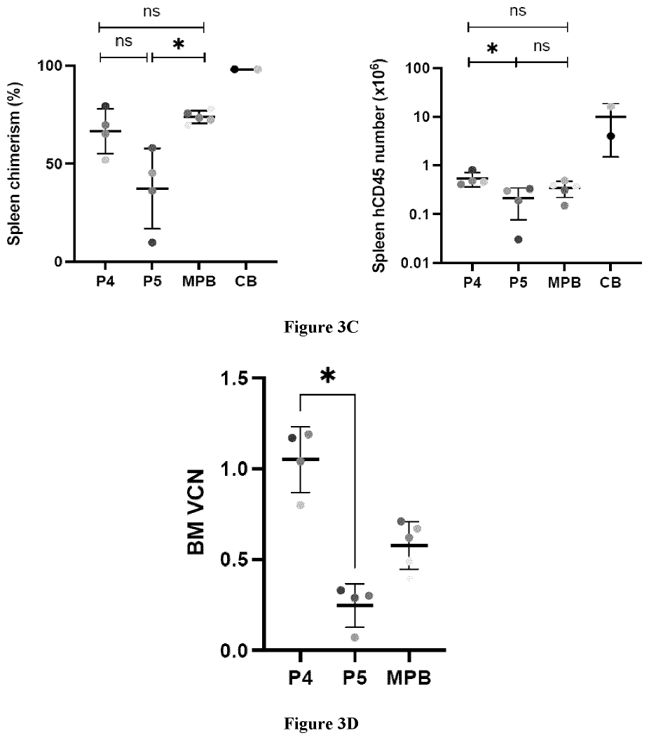

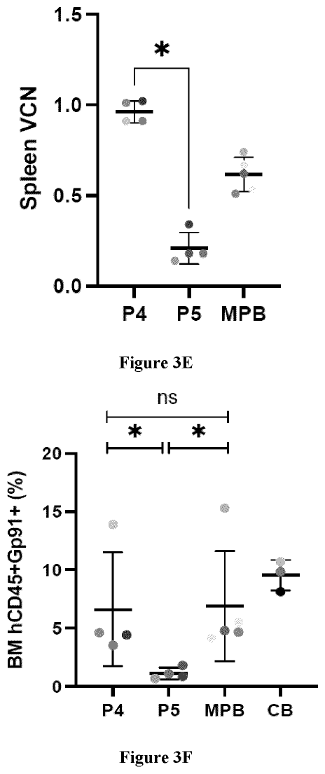

Using an aliquot of the patient's IMP, we infused engineered HSPCs into NSG mice (P4: 4.3×10⁵ cells; P5: 3.5×10⁵ cells; n=4 mice per patient). As controls, we infused nontransduced cord blood (CB) (2.7×10⁵ cells, n=3) and a sample of MPB transduced with same protocol as for the IMP (3.7×10⁵ cells, n=5). We then analyzed engraftment in the BM and spleen after 16 weeks. The mean level of BM chimerism was 35% in P5 recipients and 60% in P4 recipients; this difference was statistically significant (p=0.0286, FIG. 3A). P5 recipients had a lower absolute human CD45+ (hCD45) cell count than P4 recipients, although the difference did not reach statistical significance. Human BM HSPCs (defined as CD45+CD34+ cells) were also significantly less frequent in P5 recipients than in P4 recipients (FIG. 3B). These results were confirmed by a chimerism analysis of the spleen (FIG. 3C). Although P4 and P5 recipients had similar levels of gene correction in the IMP (1.73 and 1.58, respectively), the VCN in hCD45 cells from BM and spleen was significantly lower after transplantation-especially for P5 recipients (a mean value of 0.25, vs. 1.05 for P4; p=0.0286) (FIG. 3D-E). Similarly, the level of correction (estimated from the Gp91 protein expression by the hCD45 cells) was significantly lower in P5 recipients than in P4 recipients (p=0.0286, FIG. 3F).

These in vivo experiments demonstrated that P5's HSPC had a lower engraftment ability and gp91 expression than their counterparts from P4; this finding was in line with the corresponding clinical outcomes in the GT trial.

Taken as a whole, our results showed that P5's HSPCs presented with a chronic inflammatory profile and molecular alterations that strongly impaired their functional capacity.

### Discussion:

Our present results showed that severe interferon score profoundly alter HSC and compromise GT efficiency for CGD patients. GT remains a potentially curative treatment option for patients with CGD who lack an HLA-compatible donor for HSCT, and do not present exacerbated inflammatory marks. We observed the engraftment of gene-corrected cells in two patients (P1 and P4) at a level that was sufficient for clinical benefit, including complete remission in P4. The significant, stable correction of HSPCs has been maintained for more than 4 years now and is correlated with neutrophilic NADPH oxidase activity. In contrast—as also mentioned for four children in another clinical trial of GT (Kohn et al., 2020)—P2 and P5 progressively lost the corrected cells; the results of an in-depth single-cell transcriptomic analysis suggested that this defect might be linked to (i) a high inflammation score in the most immature HSCs, and (ii) the upregulation of specific biomarkers.

The fact that HLA-identical HSCT gives excellent outcomes in patients with CGD (i.e. low graft failure and mortality rates) suggests that the HSC niche in the BM microenvironment is not significantly altered. In a multicenter study of allo-HSCT in 712 patients with CGD, the estimated overall survival and event-free survival rates at 3 years were 85.7% and 75.8%, respectively (Chiesa et al., 2020). These results suggest that exposure to chronic inflammation caused an intrinsic HSC alteration. Using single-cell transcriptome profiling, we identified specific inflammatory signatures (including IFNγ and IFNα responses) in CGD HSCs and myeloid progenitors (monocytes, dendritic cells and neutrophils). Moreover, the two patients with the highest inflammation scores present a high frequency of myeloid progenitors and a low frequency of immature HSCs.

Our results are in line with the increased myeloid differentiation observed in response to various inflammatory emergency signals, such as IL1 (Pietras et al., 2016), IFN, and lipopolysaccharide (Chen et al., 2010; Takizawa et al., 2017; Zhang et al., 2016). Indeed, microbial infections and other stimuli (e.g. metabolic stress) can drive HSCs out of dormancy and favor proliferation and myeloid differentiation (facilitating the host's defense). This emergency granulopoiesis is initiated by the key transcription factor CEBPβ (Hirai et al., 2006). Signaling through G-CSF and STAT3 can induce a switch from CEBPα-dependent steady state granulopoiesis to CEBPβ-dependent emergency granulopoiesis (Manz & Boettcher, 2014). The impact of persistent inflammation has also been reported in a mouse model of X-CGD, with greater HSC proliferation and differentiation toward myeloid lineages (Weisser et al., 2016). Our P2 displayed an expansion of both MonoDC progenitors and B cell progenitors, which share a number of markers. These findings are reminiscent of the pro-B cell progenitor expansion that occurs after IFN stimulation (Montandon et al., 2013) and the reprogramming of myeloid lineages in a context of inflammation (Audzevich et al., 2017; Xie et al., 2004).

Even though acute inflammation can be beneficial, it is known that long-lasting, chronic inflammation strongly impairs HSC function. Whereas acute treatment with IFNα can promote the proliferation of murine HSCs, chronic IFNα activation compromises the HSCs' repopulating activity (Essers et al., 2009). Several studies have shown that elevated interferon signaling in the context of chronic infection is the major cause of HSC exhaustion and depletion (Essers et al., 2009; King et al., 2011; Lin et al., 2014; Matatall et al., 2016; Sato et al., 2009).

A pathway analysis of the most immature HSCs indicated that the higher interferon score in P2 and P5 might be responsible for the loss of gene-corrected cells and for HSC exhaustion. In contrast, the intermediate level of interferon pathway activity in P1 and P4 might have helped to maintain a beneficial response and the HSCs' repopulating ability. By taking advantage of regularized logistic regression and the large number of cells provided by single-cell analyses, we identified a set of 51 interferon genes and transcription factors that were upregulated specifically in P2 and P5 (including IFI44L, STAT2, IRF9, MX1, SAMD9L, and CEBPB) and that appeared to be predictive of defective HSC engraftment. These transcriptomic alterations and biomarkers appear to be specific to the HSC compartment, as no significant differences in the inflammation score were observed in PBMCs.

It has been shown that interferon pathway activation in HSCs involves STAT1 and IRF9 signaling (Baldridge et al., 2010) through the formation of the DNA-binding STAT1-STAT2-IRF9 ternary complex ISGF3, which then activates ISGs (Crow & Stetson, 2021). The strong activation of the interferon pathway observed in patients with CGD resulted in marked overexpression of the ISGF3 complex-especially in P2. The latter patient displayed a high frequency of monocyte/dendritic cell progenitors with strong inflammatory profile but also the upregulation of several stress-induced factors (such as JunD or SREBF1) in HSCs, which might have been responsible for the functional defects (Lu et al., 2022; Roy et al., 2021). This situation was reminiscent of HSC exhaustion through chronic IFN pathway activation (Zhang et al., 2016).

P5 had a large neutrophil progenitor population and aberrant expression of CEBPβ very early in the HSC differentiation process. The epigenetically inscribed infection history is known to make HSCs more responsive to secondary stimulation (de Laval et al., 2020). However, chronic lipopolysaccharide stimulation drives HSC exhaustion and dysfunction (Esplin et al., 2011). We hypothesize that in P2 and P5, chronic interferon stimulation epigenetically blocked HSCs in an aberrant state and thus drove exhaustion. One of the downstream markers observed in both patients was sterile a motif domain-containing protein 9-like encoded by SAMD9L, an ISG-induced gene in which mutations are known to predispose to pancytopenia and myeloid malignancies. Indeed, gain-of-function (GOF) mutations in SAMD9L have been reported in people with ataxia pancytopenia syndrome (Davidsson et al., 2018). The antiproliferative effect of these GOF mutations led to greater DNA damage and apoptosis (responsible for BM hypocellularity). A secondary mutation (monosomy 7) would favor the development of myelodysplastic syndromes (Thomas et al., 2021). Enhanced expression of SAMD9L (correlating with the higher interferon scores in P2 and P5) might therefore contribute to HSC exhaustion in a context of chronic interferon activation.

The sometimes poor transduction ability in CGD HSPCs also prompted us to optimize the transduction procedure by adding PGE2; this adjuvant is known to favor HSC homing, survival, proliferation, and repopulation ability (Hoggatt et al., 2009; North et al., 2007; Zonari et al., 2017). PGE2's proinflammatory role during vasodilatation, vascular leakiness and pain has long been known (Chace et al., 1995) but this compound can also mediate anti-inflammatory effects (Weissmann, 1993), as also shown in our transcriptomic analysis. Despite its indubitable benefit, this short course of PGE2 was not enough to counter the HSC alteration induced by chronic inflammation in P2 and P5. Moreover, PGE2 does not completely restore transduction efficiency, which is lower than in HDs-probably due to the upregulation of restriction factors like MX1, MX2 and IFITM3 (Colomer-Lluch et al., 2018; Goujon et al., 2013). The expression of these factors by HSCs during an innate immune response inhibited lentiviral entry but could be overcome by exposure to cyclosporine H (Petrillo et al., 2018) or other transduction enhancers; this aspect might be important in the further development of GT for inflammatory diseases.

Chronic inflammation in CGD might eventually favor the emergence of mutated clones with a proliferative advantage; in turn, this might lead to tumor events (Jofra Hernández et al., 2021) and so further highlights the need to control hyperinflammation.

The impaired repopulating ability of CGD HSCs has been previously reported in a mouse model of X-CGD exposed to a high IL1 concentration. Pre-treatment of X-CGD mice with anakinra (an IL1R antagonist) improves HSC engraftment (Weisser et al., 2016). More recently, p38MAPK (a downstream target of IL1β was identified in a CRISPR Cas9 screening step as a druggable target for increasing HSC engraftment. Ex vivo culture of CGD HSPCs in the presence of a p38MAPK inhibitor increased chimerism significantly (1.5-fold) (Klatt et al., 2020).

Inhibition of the JAK/STAT pathway would be another way to target the hyperactivated interferon pathway. Given that several studies have described encouraging results for JAK1 inhibition in type I interferonopathies (Forbes et al., 2018; Montealegre Sanchez et al., 2018; Vanderver et al., 2020), this approach could also been considered for controlling inflammation before HSPC harvesting in patient with CGD and thus for avoiding HSC exhaustion.

Together with the recently published study of GT for CGD, our present results show that GT is a potentially curative treatment option in patient with CGD lacking an HLA-compatible donor. However, the specific clinical and cellular characteristics of good candidates for GT (notably with regard to the inflammatory background) need to be taken into account. Our present study identified a interferon-pathway-related transcriptional signature that was specific to HSCs from patients with poor engraftment. The present results might open the way to (i) specific anti-inflammatory treatments for patients prior to HSPC harvesting, (ii) the optimization of ex vivo HSPC engineering, and (iii) identification of predictive biomarkers for validating the GT product.

### REFERENCES

Throughout this application, various references describe the state of the art to which this invention pertains. The disclosures of these references are hereby incorporated by reference into the present disclosure.

1. A method of assessing exhaustion of a population of hematopoietic stems cells (HSCs) obtained from a subject and treating the subject, comprising
determining the expression level of one or more genes selected from the group consisting of: IFI44L, STAT2, IRF9, MX1, SAMD9L, CEBPB, BRD7, CD69, EGR1, GBP2, H1FX, HLA-B, HLA-DQA1, ISG15, ISG20, JUND, LAP3, LGLALS3BP, LMO2, LY6E, MLF1, NCOA7, NR4A1, NR4A2, SETBEP1, TAF10, TCF4, TOP2B, TSC22D3, VAMP8, YBX3, ZNF385D, ZSCAN31, ENO1, HIST2H2BE, SREBF1, BAZ2B, BTG2, JUNB, MAFG, NFIB, ZNF439, PARP14, SPEN, MBD2, BTG1, CBX6, EIF2AK2, PSIP1, PURA, SAMD9, CASP8, CD74, HDGF, LYL1, SND1, ZBTB20, MBD3, MX2, ARL4A, DEK, PBXIP1, PQLR2L, TNFSF10, ID3, KLF12, MAF1, TRIL22, FOSL1, OAS1, TOB1, ZNF544, MLLT3, PAWR, and ZNF618
determining that the expression level of the one or more genes is higher than a corresponding predetermined reference value, and
administering an anti-inflammatory drug to a subject determined to have an expression level that is higher than the corresponding predetermined reference value.

2. The method of claim 1 wherein the one or more genes is selected from the group consisting of IFI44L, STAT2, IRF9, MX1, SAMD9L, and CEBPB.

3. The method of claim 2 wherein the one or more genes is selected from the group consisting of IFI44L, STAT2, IRF9, MX1, and SAMD9L.

4. The method according to claim 1 that comprises the steps of
i) determining the expression level of the one or more genes in the population of HSCs and
ii) comparing the expression level with the corresponding predetermined reference value, wherein a difference between the expression level determined at step i) and the corresponding predetermined reference value is indicative of whether said population of HSCs is exhausted.

5. The method of claim 4 wherein when the expression level is higher than the corresponding predetermined reference value, then it is concluded that the population of HSCs is exhausted and when the expression level is lower than the predetermined reference value, then it is concluded that the population of HSCs is not exhausted.
